(19) United States
(12) Patent Application Publication
(10) Pub. No.: US 2025/0198973 A1
(43) Pub. Date: Jun. 19, 2025

(54) POST CONCENTRATION TOTAL ORGANIC FLUORIDE MEASUREMENT

(71) Applicant: Hach Company, Loveland, CO (US)

(72) Inventors: Vishnu Rajasekharan, Fort Collins, CO (US); Scott Alan Tucker, Fort Collins, CO (US); Darren Kent MacFarland, Windsor, CO (US); Russell Martin Young, Fort Collins, CO (US)

(21) Appl. No.: 18/983,811

(22) Filed: Dec. 17, 2024

Related U.S. Application Data
(60) Provisional application No. 63/611,866, filed on Dec. 19, 2023.

(51) Int. Cl. G01N 31/12; G01N 1/38; G01N 33/00

(52) U.S. Cl. CPC G01N 31/12; G01N 1/38; G01N 33/0049

(57) ABSTRACT

An embodiment provides a method for measuring an amount of total organic fluoride content of a PFAS containing sample, including: placing a sample comprising a PFAS compound in a measurement device; introducing the sample into a combustion tube, the combustion tube comprising a catalyst; oxidizing the PFAS, using heat and the catalyst, to produce a combusted sample of a plurality of measurable simpler compounds; adjusting, using a valve, a moisturization of the combusted sample; and measuring, using the measurement device, an amount of total organic fluoride of the sample after the oxidation. Other aspects are described and claimed.

### CROSS REFERENCE TO RELATED APPLICATION

This application claims priority to U.S. Provisional Patent Application Ser. No. 63/611,866, filed on Dec. 19, 2023, and entitled “POST CONCENTRATION TOTAL ORGANIC FLUORIDE MEASUREMENT,” the contents of which are incorporated by reference herein.

### FIELD

This application relates generally to measurement of the total organic and inorganic fluoride in a sample, and, more particularly, to measurement of the total organic and inorganic fluoride in a sample using a post concentration method and device.

### BACKGROUND

Ensuring water quality is critical in a number of industries such as drinking water, waste water treatment and reuse, pharmaceuticals, and other manufacturing fields. Additionally, ensuring water quality is critical to the health and well-being of humans, animals, and plants which are reliant on the water for survival. Per and polyfluoroalkyl substances (PFAS), are a large class of synthetic chemicals that impacts public health at ultra-low levels. Governments and regulating agencies have set limits on PFAS concentration. Such limits may be set upon the PFAS compounds in surface water, ground water, wastewater, biosolids, and soils. Several industries that use or have used PFOS/PFOA are replacing with alkyl fluoro substitutes which still are of concern.

### BRIEF SUMMARY

In summary, one embodiment provides a method for measuring an amount of total organic fluoride content of a PFAS containing sample, comprising: placing a sample comprising a PFAS compound in a measurement device; introducing the sample into a combustion tube, the combustion tube comprising a catalyst; oxidizing the PFAS, using heat and the catalyst, to produce a combusted sample of a plurality of measurable simpler compounds; adjusting, using a valve, a moisturization of the combusted sample; and measuring, using the measurement device, an amount of total organic fluoride of the sample after the oxidation.

Another embodiment provides a device for measuring an amount of total organic fluoride content of a PFAS containing sample, comprising: a combustion tube; a catalyst; a moisturizer; and a processor; the device for measuring an amount of fluoride content of a PFAS compound in a sample being configured to: placing a sample comprising a PFAS compound in a measurement device; introducing the sample into the combustion tube, the combustion tube comprising the catalyst; oxidizing the PFAS, using heat and the catalyst, to produce a combusted sample of a plurality of measurable simpler compounds; adjusting, using a valve, a moisturization of the combusted sample; and measuring, using the measurement device, an amount of total organic fluoride of the sample after the oxidation.

A further embodiment provides a product for measuring an amount of total organic fluoride content of a PFAS containing sample, comprising: a storage device having code stored therewith, the code being executable by a processor and providing instructions to: introduce the sample into the combustion tube, the combustion tube comprising the catalyst; oxidize the PFAS, using heat and the catalyst, to produce a combusted sample of a plurality of measurable simpler compounds; adjust, using a valve, a moisturization of the combusted sample; and measure, using the measurement device, an amount of total organic fluoride of the sample after the oxidation.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example device for total organic fluoride measurement.

FIG. 2 illustrates a flow diagram for total organic fluoride measurement.

FIG. 3 illustrates another example flow diagram for total organic fluoride measurement.

FIG. 4 illustrates example data of total organic fluoride measurement from recovery of fluoride from a PFBS digestion.

FIG. 5 illustrates an example of computer circuitry.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

Per and polyfluoroalkyl substances (PFAS) are a large class of synthetic chemicals that impacts public health at ultra-low levels, even in the range of part-per-trillion (ppt). The current Environmental Protection Agency in the United States health advisory states 4 ng/L maximum for PFOS and PFOA in drinking water and recommends total organic fluoride (TOF) at 1 μg/L as a surrogate measurement of fluorine compounds in surface water, ground water, wastewater, biosolids and soils. Other governments have mandated regulatory limits for PFAS compounds. Significant investment is made by treatment plants to achieve these limits. Many of these treatment methodologies are not entirely validated. Several industries that used PFOS/PFOA are replacing with alkyl fluoro substitutes, and these replacements remain a health and environmental concern. Very few high-end lab methods are approved by agencies for PFAS analysis. No technologies exist for analyzing PFAS in field settings. In many cases field analysis of these parameters is impractical due to the complex sample preparation needed in the existing methods.

There are many challenges to TOF measurement which may be critical for detection of per and polyfluoroalkyl substances. For example, a method and system require an ultra-low-level detection of a diverse set of compounds. The PFAS compounds have a widespread presence in the environment. The difficulty of degradation of larger molecules to recalcitrant smaller ones, precursor transformations, changes in the chemical structure after discharge from an industry or during the leaching process from a landfill, and high background during the analysis of TOF since most tap water has 0.5 to 1 ppm inorganic fluoride anion background from city treatment. Additionally, there are a wide variety of locations and conditions to be tested such as military bases, airports, industrial plants, landfills, firefighting training sites, or the like. Some tests only measure a limited set if targeted chemical and precursors, and fail to identify all PFAS compounds.

Specifically, some methods to detect PFAS compounds have limitations. For example, combustion ion chromatography may have inefficient adsorption of organic and inorganic fluoride activated carbon. The method may have the added step to elute inorganic fluoride eluted from the activated carbon by washing with neutral nitrate solution. There remains a complex process of burning the adsorbable organic fluoride (AOF) loaded activated carbon burnt in an oxygen stream under pyro hydrolytic conditions. This may produce toxic gaseous byproducts of the combusted activated carbon trapped in an absorption medium where corrosive hydrogen fluoride is formed during combustion process. The corrosive hydrogen fluoride is then condensed back into the solution that dissociates to form fluoride anion. A separate aliquot of known volume of the absorbing solution that contains fluoride anion is then injected into an ion chromatograph that is complex in nature by means of a sample injection valve. This requires long cycle times for the halide anions to be separated on the anion separation column of the ion chromatograph (IC). Additionally, conductivity of the eluent needs to be reduced with an anion suppression device prior to the IC conductivity detector where fluoride is measured.

What is needed is an accurate and simpler method for TOF measurement for PFAS analysis. For example, to eliminate the combustion cartridge, and achieve in-situ digestion of PFAS and post concentration of fluoride ions. Also, complete digestion of PFAS compounds by combusting the sample directly in a mini furnace without any cartridge or reagents. Efficiency of digestion may be maximized by the optimizing the amount of sample combusted in the presence of catalyst and transporting the combusted gas into a predetermined volume of deionized (DI) water. PFAS Treatment efficacy validation may be achieved through this total organic fluoride, and total oxidizable precursor measurements. For example, a water processing plant may want to determine a fluoride measurement of the sample with PFAS compounds within the facility because a high concentration of fluoride is hazardous, cause damage or disease to people, and/or the like. Accordingly, the water processing plant can use the described method by providing a sample to the measurement chamber. The system then performs the measurement as per the described system. From the measurement, the water processing plant or an operator within the plant can dose, trat, or otherwise capture the water to reduce the concentration or dispersal of the fluoride or PFAS containing sample. Thus, the described system can guide the water processing plant, or other entity or user, to perform upstream actions to modify the fluoride within the water processing plant.

Accordingly, the method and device described herein provide a technique for measurement of PFAS and other substances in a sample. In an embodiment, the total organic and inorganic fluoride may be determined in the most efficient manner. For example, a sample containing PFAS may be directly introduced, aspirated, or placed into a combustion tube. Aspiration may be used for a liquid sample. However, a sample may be in a solid form and placed in the combustion tube. The combustion tube may comprise a catalyst and/or an inert material such as quartz or the like. The combustion tube may be heated to a temperature range of about 500-2000 degrees Celsius, preferably 700-1100 degrees Celsius. The temperature may ensure complete oxidation of the PFAS. A carrier gas may be added and/or harvested from ambient air or from electrochemical oxidation of water. In an embodiment, inorganic fluoride may be removed using a cartridge. A liquid or solid sample with PFAS may be completely vaporized, and the gases generated may be moisturized by flowing the combusted gas over the DI water. Alternatively, a combusted dry gas sample may be added to a known amount of DI water. Moisturization may allow for in-situ post moisturization of the oxidized sample. A flow rate may be adjusted to control the gas flow over the DI water to control an extent of moisturization. A sample flow rate may be adjusted to control and separate an amount of water vapor generated. In an embodiment, the catalyst may be conditioned and reactivated using a regeneration reagent. Measurement of the TOF may be performed using electrochemical, colorimetric, chromatographic methods, or the like.

The illustrated example embodiments will be best understood by reference to the figures. The following description is intended only by way of example, and simply illustrates certain example embodiments.

In an embodiment, the described method and system may simplify the total organic fluoride analysis. For example, elimination of the combustion cartridge, and hence avoiding the production of hydrogen fluoride gas in the existing total organic fluoride (TOF). The system and method may provide for in-situ digestion of PFAS. In an embodiment, a post concentration of fluoride ions may be achieved. The method and device may completely digest PFAS by combusting the sample in a mini furnace, or combustion tube, without any cartridge and/or reagents. An efficiency of digestion may be maximized by optimizing the amount of sample combusted or oxidized in the presence of a catalyst. In an embodiment, the combusted gas may be transported into a predetermined volume of DI water. In an embodiment, the combusted gas may be flowed through a moisturizer module at a controlled flow rate to achieve a desired post concentration.

For ease of reading the disclosure mainly focuses upon PFAS compound. However, the method and device described herein may be used to measure other compounds for which a TOF measurement may be required. It is understood that facilities, regulatory agencies, and other industries require measurement of samples containing compounds similar to PFAS compounds.

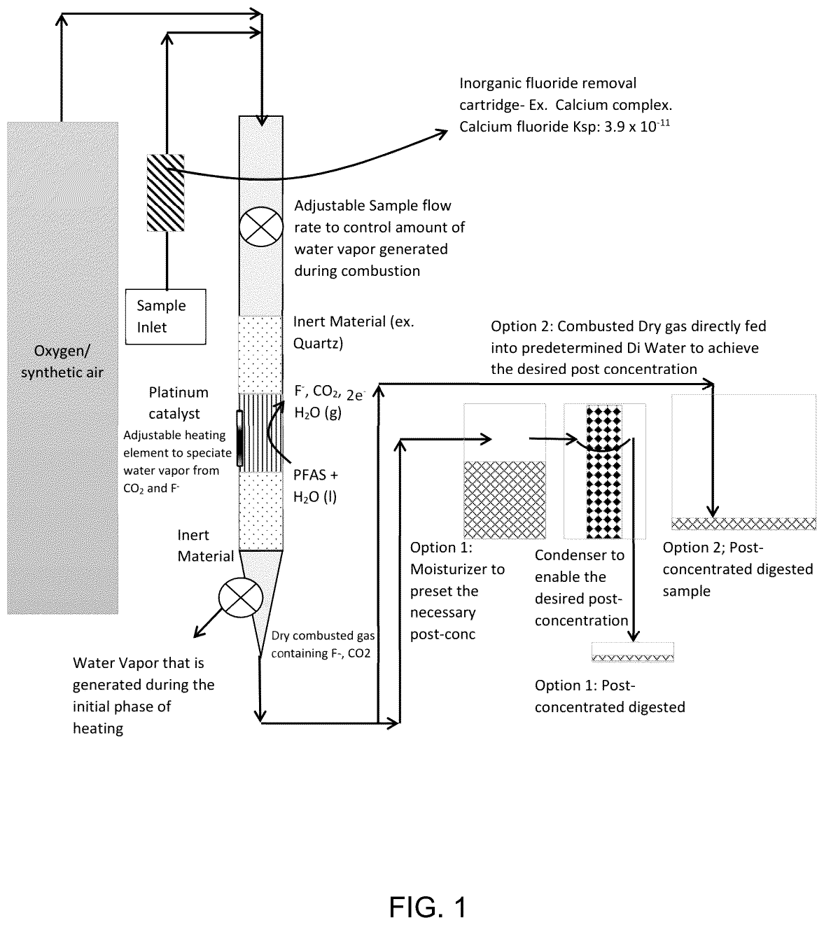

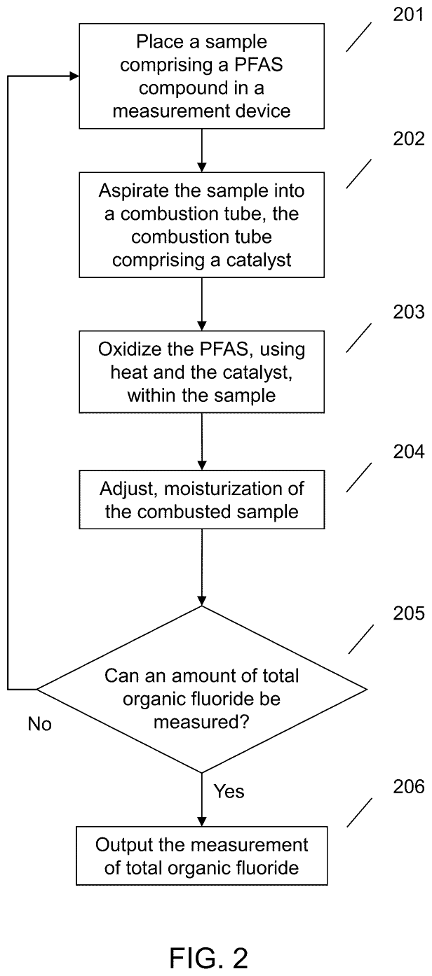

Referring to FIG. 1, in an embodiment, an example device for measurement of total organic fluoride (TOF) is illustrated. The device of FIG. 1 is an example device to illustrate the method steps of an embodiment illustrated in FIG. 2. Referring to FIG. 2 at 201, in an embodiment a sample may be placed in a measurement device. The sample may contain a PFAS compound or an amount of a PFAS compound. The sample may be drinking water, industrial effluent, from a natural body of water, from a holding tank, a municipal or industrial waste water source, or the like. The sample may be pumped, gravity fed, placed by a user, or the like. An introduction of a sample may be manual or automated, and may be set to periodic measurement periods of time. In an embodiment, the sample containing a PFAS compound may be introduced into the device via a sample inlet. The sample inlet may be upstream of a combustion tube.

In an embodiment, a carrier gas may be added to the sample. The carrier gas or gas may be oxygen, synthetic air, ambient air, or the like. For example, oxygen or synthetic air may be harvested from ambient air. As another example, oxygen or synthetic air may be produced by electrochemical oxidation of water. The carrier gas or gas may be used for digestion, oxidation, and/or transport of combusted gas as described below. In an embodiment, the carrier gas may be introduced with a sample prior to the sample entry to the combustion tube. In an embodiment, the carrier gas may be stored in a cylinder or vessel, or generated as required. The gas may be pumped, moved via pressure differential, or the like. The control of the flow of gas may be controlled by one of more valves, and one of more sensors for pressure, flow, gas concentration, or the like. The valves and sensors may be operatively coupled to a system to control an optimal flow or delivery of gas at a proper time.

At 202, in an embodiment, a sample may be introduced to a combustion tube. In an embodiment, the sample may be aspirated into the combustion tube. The sample may be placed by a user, pumped, gravity fed, or the like into the combustion tube. In an embodiment, the combustion tube may comprise an inert material. The inert material may be quartz. The inert material may properly space components of the combustion tube and/or allow a spacing both spatially and thermally between combustion tube components. In an embodiment, the combustion tube may have a catalyst centered between two regions of inert material. In other words, a sample may enter the combustion tube and travel through an inert region, then a catalyst, and then another inert region. In an embodiment, the combustion tube comprises a catalyst. In an embodiment, the catalyst may be platinum, rhodium, or the like. In an embodiment, a stable platinum black material may be used. For example, the catalyst may be spherical platinum black coated alumina, nickel, or the like.

In an embodiment, a cartridge may be placed between the sample inlet and the combustion tube. The cartridge may be an inorganic fluoride removal cartridge. For example, a calcium complex containing cartridge may be used (i.e. calcium fluoride Ksp 3.9×10⁻¹¹). The fluoride content measured before the oxidation provides the total inorganic fluoride (TIF). Removal of inorganic fluoride using calcium or other alkali metal cartridges. In an embodiment, the inorganic fluoride may be measured by the system or method. Periodic maintenance and/or measurement of the cartridge performance may be performed to maintain inorganic fluoride removal.

At 203, in an embodiment, the PFAS of the sample may be oxidized in the combustion tube with a catalyst and heat. In an embodiment, the combustion tube comprises a heating element. The heating element may be adjustable to a predetermined or selected temperature. The heating element may be adjusted to speciate water vapor from carbon dioxide and/or fluoride. The heating rate may be adjusted to control and separate the amount of water vapor generated. For example, a moisture sensor downstream of the heating element may provide information to the system to adjust the heating element to cause a change in water vapor generation.

The heating element may heat the catalyst. The temperature of the catalyst may be heated to range of about 500-2000 degrees Celsius, preferably 700-1100 degrees Celsius. The temperature range may completely oxidize the PFAS compound. The combustion tube allows for a homogeneous heat transfer with efficient temperature distribution. In other words, the heating element and catalyst may be thermally coupled to each other, and thermally isolated by the inert material at either end of the catalyst. The catalyst assists with the reaction of a PFAS compound and liquid water to a fluoride, carbon dioxide, and water gas product. The oxidation of the PFAS, using heat and the catalyst, may produce a combusted sample of a plurality of measurable simpler compounds.

In an embodiment, the catalyst may be reactivated. The reactivation may be referred to as conditioning. For example, the platinum catalyst and/or ceramic pad may be treated with silver nitrite. In an embodiment, another regeneration reagent may be used. The method or system may use sensors to measure oxidation efficiency, use a period of time, duty cycles, amount of TOF measured, or the like to determine when a catalyst requires regeneration.

In an embodiment, the combustion tube may have a valve at the proximal end (sample inlet side), and may have a valve at the distal end (combustion gas outlet side). In an embodiment, the proximal valve may be referred to as an adjustable sample flow rate valve, or sample flow valve. The adjustable sample flow rate may control an amount of water vapor generated during combustion. For example, increasing a sample flow rate may increase the amount of water vapor generated during combustion or oxidation. Such a sample control flow may be used to produce a desired post concentration of the oxidized sample or oxidized gas.

In an embodiment, the distal valve may be referred to as a water vapor valve. For example, water vapor after the oxidation may be either retained or allowed to escape to produce a desired post concentration of the oxidized sample or oxidized gas. These valves may be operatively coupled to one or more sensors. The sensors may measure temperature, pressure, moisture content, flow rate, or the like. The sensors and valves are operatively coupled to a system to the management of sample flow, water vapor content, pressure, or the like such that measurement, feedback, adjustment, and recording of parameters may be made.

At 204, in an embodiment, the method and device may adjust a moisturization of the combusted sample (See FIG. 1). The moisturization step may be accomplished in two main alternative methods. In one method, the liquid samples are completely vaporized and the gases generated are moisturized by flowing the combusted gas over a volume of DI water. There is propensity for the fluoride formed after the decomposition of the PFAS to react with the glass (SiO₂) in the combustion tube to form H₂SiF₆. This may result in loss of fluoride and degradation of the combustion tube. The sample may then be condensed to achieve a desired post-concentration.

At 204, in another embodiment, the method and device may adjust a moisturization of the combusted sample (See FIG. 1). In this alternative method, the combusted dry gas may be directly fed into a predetermined amount of DI water to achieve a desired post concentration. In an embodiment, post concentration is the process of concentrating a digested simpler form of an analyte since the analyte may not be measured by the technique in a desired concentration range. For both methods of moisturization, the valves on the combustion tube, and sensors capable of measuring moisture content throughout the sample flow may be used to adjust moisturization of the sample and/or combusted sample.

The method and system, at 205, may determine an amount of TOF of the sample. If, however, the TOF may be determined at 205, the system, at 206, may output an amount of total organic fluoride, a PFAS concentration, or the like of the sample. The system may also output parameters such as flow rate of a sample or gas, amperage, heating element settings, time of digestion, catalyst performance, TOF and TIF measurement, or the like. In an embodiment, an output may be in the form of a display, storing the data to a memory device, sending the output through a connected or wireless system, printing the output, or the like. The system may be automated, meaning the system may automatically output a measurement. The system may also have associated alarms, limits, or predetermined thresholds. For example, if a measured value reaches a threshold, the system may trigger an alarm, alert the system/personnel to a fault, alter the flow of the sample, or the like. Data may be analyzed in real-time, stored for later use, or any combination thereof. At 205, if an amount of TOF cannot be determined, the system may obtain another sample for testing, output an alarm, send a reminder for maintenance, shunt the flow of sample, or the like. Measurement of TOF/TIF, or the like may be performed by electrochemical, colorimetric, or chromatographic methods.

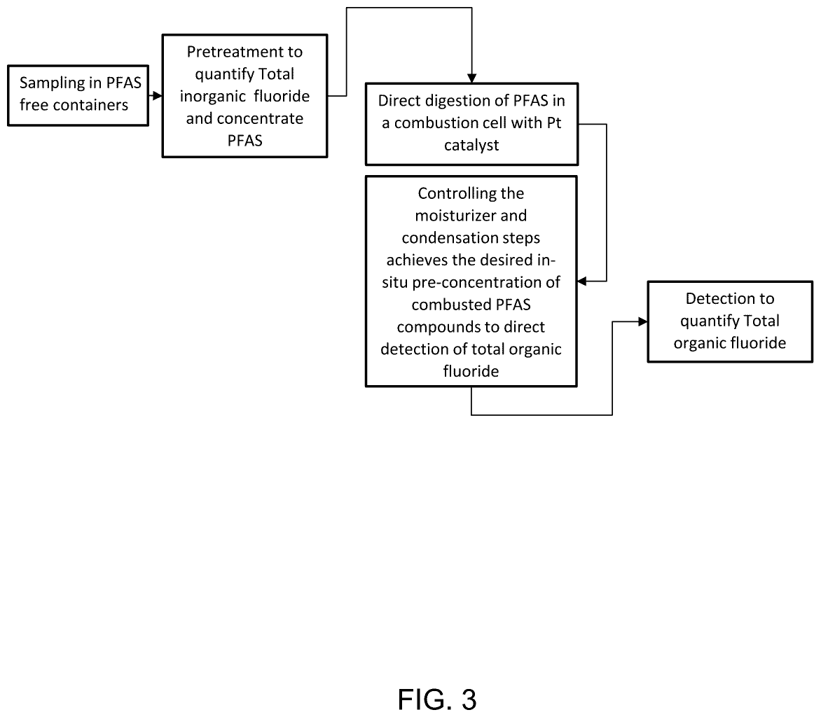

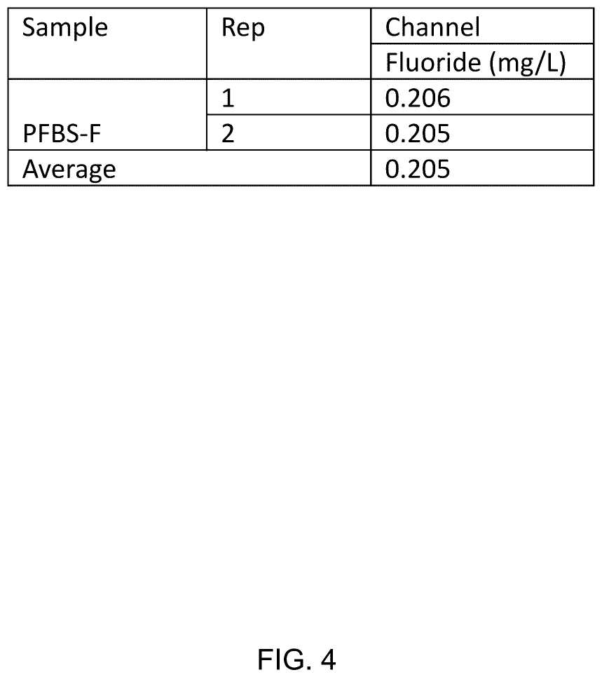

Referring to FIG. 3, in an embodiment, another example flow diagram for total organic fluoride measurement is illustrated. For example, a workflow of direct digestion and preconcentration is shown. In an embodiment, a sample may be pretreated to qualify TIF and concentrate PFAS in a sample. The method may then directly digest PFAS in a combustion cell using a platinum catalyst. The method may then control moisturization and condensation steps to achieve a desire in-situ pre-concentrate of combusted PFAS compound to direct detection of TOF. Referring to FIG. 4, exemplar data from the method of FIG. 3 is illustrated. In an embodiment, an 82% recovery rate is observed for Perfluorobutanesulfonic acid (PFBS).

The aqueous sample may be placed or introduced into a cartridge, digestion cell, or the like manually by a user or using a mechanical means, for example, gravity flow, a pump, pressure, fluid flow, or the like. For example, a water sample for testing may be introduced to a chamber by a pump. In an embodiment, there may be one or more chambers in which the one or more method steps may be performed. In an embodiment, valves or the like may control the influx and efflux of the sample into or out of the one or more chambers, if present. Once the sample is introduced to the measurement system, the system may measure a sample automatically.

The sample may be placed or introduced into the combustion tube, or any vessel or tubing of the device, or the like manually by a user or using a mechanical means, for example, gravity flow, a pump, pressure, fluid flow, or the like. For example, a water sample for testing may be introduced to a chamber by a pump. In an embodiment, there may be one or more chambers in which the one or more method steps may be performed. In an embodiment, valves or the like may control the influx and efflux of the sample into or out of the one or more chambers, if present. Once the sample is introduced to the measurement system, the system may measure a sample automatically.

The various embodiments described herein thus represent a technical improvement to conventional methods and instrument for PFAS, fluoride, or other analyte measurement. Using the techniques as described herein, an embodiment may use a method and device for an instrument for PFAS measurement. This is in contrast to conventional methods with limitations mentioned above. Such techniques provide a better method and an instrument for PFAS measurement.

Some advantages to the method and device described herein as opposed to conventional techniques are listed herein. Direct combustion of PFAS sample that leads to true TOF rather than adsorbable organic fluoride (AOF) or extractable organic fluoride (EOF). By measuring the fluoride content before the oxidation, the total inorganic fluoride is provided. The inorganic fluoride is removed using calcium or other alkali metal cartridges. The post concentration of fluoride occurs through the moisturizer and condensation steps.

The combustion, moisturizer, condensation steps provide the following advantages listed below as compared to conventional techniques. These steps ensure completion of oxidation for each concentration and type of PFAS compound. The steps provides the ability to determine the background contribution that can occur due to inefficient upstream inorganic fluoride removal. Post concertation duration can be optimized for each run that is dependent on the concentration and type of PFAS. These steps ensure the oxidation process and the oxidation cell integrity by monitoring the fluoride, CO2, and pH in the post concentrated fluoride measuring cell. Use of these steps provide dynamic control of the post concentration steps to achieve reliable detection of the fluoride after PFAS combustion. The steps are able to provide an analysis of the fluoride generation profiles to classify the type of PFAS compounds. The use of those steps provide controlled heating and sample flow to separate and control water vapor to achieve the desired post concentration.

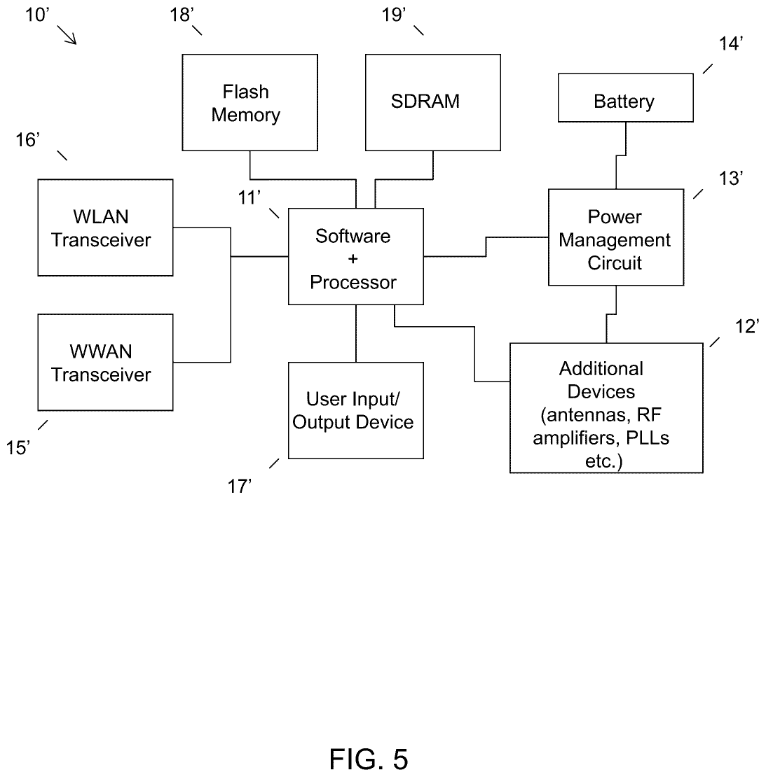

While various other circuits, circuitry or components may be utilized in information handling devices, with regard to an instrument for fluoride measurement according to any one of the various embodiments described herein, an example is illustrated in FIG. 5. Device circuitry 10′ may include a measurement system on a chip design found, for example, a particular computing platform (e.g., mobile computing, desktop computing, etc.) Software and processor(s) are combined in a single chip 11′. Processors comprise internal arithmetic units, registers, cache memory, busses, I/O ports, etc., as is well known in the art. Internal busses and the like depend on different vendors, but essentially all the peripheral devices (12′) may attach to a single chip 11′. The circuitry 10′ combines the processor, memory control, and I/O controller hub all into a single chip 11′. Also, systems 10′ of this type do not typically use SATA or PCI or LPC. Common interfaces, for example, include SDIO and I2C.

There are power management chip(s) 13′, e.g., a battery management unit, BMU, which manage power as supplied, for example, via a rechargeable battery 14′, which may be recharged by a connection to a power source (not shown). In at least one design, a single chip, such as 11′, is used to supply BIOS like functionality and DRAM memory.

System 10′ typically includes one or more of a WWAN transceiver 15′ and a WLAN transceiver 16′ for connecting to various networks, such as telecommunications networks and wireless Internet devices, e.g., access points. Additionally, devices 12′ are commonly included, e.g., a transmit and receive antenna, oscillators, PLLs, etc. System 10′ includes input/output devices 17′ for data input and display/rendering (e.g., a computing location located away from the single beam system that is easily accessible by a user). System 10′ also typically includes various memory devices, for example flash memory 18′ and SDRAM 19′.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data. The disclosed system may be used in an embodiment of an instrument for fluoride measurement.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a measurement device such as illustrated in FIG. 1, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for measuring an amount of total organic fluoride content of a PFAS containing sample, comprising:
placing a sample comprising a PFAS compound in a measurement device;
introducing the sample into a combustion tube, the combustion tube comprising a catalyst;
oxidizing the PFAS, using heat and the catalyst, to produce a combusted sample of a plurality of measurable simpler compounds;
adjusting, using a valve, a moisturization of the combusted sample; and
measuring, using the measurement device, an amount of total organic fluoride of the sample after the oxidation.

2. The method of claim 1, further comprising feeding the combusted sample to a predetermined amount of distilled water to achieve a desired post concentration.

3. The method of claim 1, further comprising adjusting, using a valve and a moisture sensor, a moisturization of the sample.

4. The method of claim 3, wherein the adjusting the moisturization produces a desired post concentration of the oxidized sample.

5. The method of claim 1, wherein the catalyst is selected from the group consisting of: platinum and rhodium.

6. The method of claim 1, further comprising removing, using a cartridge, inorganic fluoride from the sample prior to the digesting, wherein the cartridge precipitates the inorganic fluoride.

7. The method of claim 1, wherein the adjusting the flow of the sample, using a sample flow valve, controls an amount of water vapor generated in the combustion tube.

8. The method of claim 1, wherein the combustion tube further comprises an adjustable heating element.

9. The method of claim 1, further comprising periodically reactivating the catalyst with a regeneration reagent.

10. The method of claim 1, further comprising adding a carrier gas to the sample prior to the oxidation.

11. A device for measuring an amount of total organic fluoride content of a PFAS containing sample, comprising:
a combustion tube;
a catalyst;
a moisturizer; and
a processor;
the device for measuring an amount of fluoride content of a PFAS compound in a sample being configured to:
placing a sample comprising a PFAS compound in a measurement device;
introducing the sample into the combustion tube, the combustion tube comprising the catalyst;
oxidizing the PFAS, using heat and the catalyst, to produce a combusted sample of a plurality of measurable simpler compounds;
adjusting, using a valve, a moisturization of the combusted sample; and
measuring, using the measurement device, an amount of total organic fluoride of the sample after the oxidation.

12. The device of claim 11, further comprising feeding the combusted sample to a predetermined amount of distilled water to achieve a desired post concentration.

13. The device of claim 11, further comprising adjusting, using a valve and a moisture sensor, a moisturization of the sample.

14. The device of claim 13, wherein the adjusting the moisturization produces a desired post concentration of the oxidized sample.

15. The device of claim 11, wherein the catalyst is selected from the group consisting of: platinum and rhodium.

16. The device of claim 11, further comprising removing, using a cartridge, inorganic fluoride from the sample prior to the digesting, wherein the cartridge precipitates the inorganic fluoride.

17. The device of claim 11, wherein the adjusting the flow of the sample, using a sample flow valve, controls an amount of water vapor generated in the combustion tube.

18. The device of claim 11, wherein the combustion tube further comprises an adjustable heating element.

19. The device of claim 11, further comprising periodically reactivating the catalyst with a regeneration reagent.

20. A product for measuring an amount of total organic fluoride content of a PFAS containing sample, comprising:
a storage device having code stored therewith, the code being executable by a processor and providing instructions to:
introduce the sample into the combustion tube, the combustion tube comprising the catalyst;
oxidize the PFAS, using heat and the catalyst, to produce a combusted sample of a plurality of measurable simpler compounds;
adjust, using a valve, a moisturization of the combusted sample; and
measure, using the measurement device, an amount of total organic fluoride of the sample after the oxidation.
